(19) United States
(12) Patent Application Publication
(10) Pub. No.: US 2022/0114246 A1
(43) Pub. Date: Apr. 14, 2022

(54) Protected sharing of data saved in a hearing device

(71) Applicant: SONOVA AG, Staefa (CH)

(72) Inventors: Nadim El Guindi, Bassersdorf (CH); Anne Thielen, Stäfa (CH); Andreas Breitenmoser, Zurich (CH); Harald Krueger, Affoltern am Albis (CH)

(73) Assignee: Sonova AG

(21) Appl. No.: 17/463,067

(22) Filed: Aug. 31, 2021

(30) Foreign Application Priority Data
Oct. 8, 2020 (EP) 20200680.5

(51) Int. Cl. G06F 21/32; H04L 29/06; G06F 21/60; H04R 25/00

(52) U.S. Cl. CPC G06F 21/32; H04R 25/554; G06F 21/606; H04L 63/0435

(57) ABSTRACT

A method for a protected sharing of data saved in a hearing system, which comprises at least a hearing device used by a hearing device user, with a third party device is presented. The method comprises: storing a definition of a first trusted party and of a second trusted party in the hearing system; providing a sharing request for the data to be shared to a device of the first trusted party and to a device of the second trusted party; generating a first sharing approval by the device of the first trusted party and a second sharing approval by the device of the second trusted party; and sharing the data with the third party device upon generation of the first and the second sharing approval.

### RELATED APPLICATIONS

The present application claims priority to EP Patent Application No. 20200680.5, filed Oct. 8, 2020, the contents of which are hereby incorporated by reference in their entirety.

### BACKGROUND INFORMATION

The accompanying drawings illustrate various embodiments and are a part of the specification. The illustrated embodiments are merely examples and do not limit the scope of the disclosure. Throughout the drawings, identical or similar reference numbers designate identical or similar elements.

Hearing devices are generally small and complex devices. Hearing devices can include a processor, microphone, speaker, memory, housing, and other electronical and mechanical components. Some example hearing devices are Behind-The-Ear (BTE), Receiver-In-Canal (RIC), In-The-Ear (ITE), Completely-In-Canal (CIC), and Invisible-In-The-Canal (IIC) devices. A user can prefer one of these hearing devices compared to another device based on hearing loss, aesthetic preferences, lifestyle needs, and budget.

In many cases, modern hearing devices include sensors, e.g. accelerometers, pulse oximetry or electroencephalography sensors etc., in order to collect health related data of the user, such as data about his physical activity or hear rate. Such data is typically stored in the hearing device and may contain sensitive or private information about the physical or mental state of the user.

In certain situations, especially if a medical condition is diagnosed, it may be desirable to share the data stored in a hearing device with a health care professional. At the same time, the data should not be accessible by unauthorized parties, such as a hacker working for a life insurance company.

To protect a hearing device against various types of hacking, inter alia against unauthorized reading out of health data saved in the hearing device, EP 3 236 674 A1 discloses a hearing device provided with public key security functions. That is, a part of the hearing device of EP 3 236 674 A1 is configured as a so-called Trusted Platform Module including a secure cryptoprocessor, a random generator, as well as a protected memory for private keys, which is only readable by the cryptoprocessor, but not by the main processor. The public key of the device, the public key of a hearing device manufacturer and an operating system are saved in a storage region of a non-volatile memory which is protected against modification, i.e. in a sealed storage region.

Further, WO 2018/091079 A1 discloses a method of controlling access of a client to a service of a hearing instrument, comprising the steps of: requesting access of the client to the service of the hearing instrument by providing a client authenticator to the hearing instrument; authenticating the client based on a validation of the provided client authenticator by the hearing instrument; upon successful authentication, comparing a security level associated with the service requested by the client with a highest security level assigned to the client by the hearing instrument, and granting access if the requested security level is below or equal to the highest security level assigned to the client. The client may comprise devices such as fitting stations, hearing instruments, wireless microphones, smartphones, tablets, remote controls etc. Since the authentication methods include, for example, authentication by user gesture, the user keeps control of client access to his hearing instrument. Further, the method protects the hearing instrument from man-in-the-middle attacks during pairing, while nevertheless the access control may be implemented in a manner that requires only little resources of the hearing instrument.

### BRIEF DESCRIPTION OF THE DRAWINGS

Below, embodiments of the present technology are described in more detail with reference to the attached drawings.

FIG. 1 schematically shows a block diagram of a hearing system according to an embodiment of the present technology.

FIG. 2 shows a flow diagram for a method according to an embodiment of the present technology for a protected sharing of data saved in the hearing system of FIG. 1 with a third party.

FIG. 3 schematically shows a sequence diagram visualizing the method of FIG. 2 according to a further embodiment of the present technology involving cascaded encryption.

FIG. 4 schematically shows a sequence diagram visualizing the method of FIG. 2 according to a further embodiment of the present technology involving signed sharing permissions.

FIG. 5 schematically shows a sequence diagram visualizing the method of FIG. 2 according to a further embodiment of the present technology involving voiceprints of trusted parties.

FIG. 6 schematically shows a sequence diagram visualizing the method of FIG. 2 according to a further embodiment of the present technology involving a central server managing the sharing approvals.

The reference symbols used in the drawings, and their meanings, are listed in summary form in the list of reference symbols. In principle, identical parts are provided with the same reference symbols in the figures.

### DETAILED DESCRIPTION

The present technology relates to a method, a computer program and a computer-readable medium for a protected sharing of data saved in a hearing system, which comprises at least one hearing device used by a hearing device user, with a third party device. Furthermore, the present technology relates to a hearing system comprising at least one hearing device used by a hearing device user and optionally a connected user device, such as a smartphone.

It is a feature of the present technology to further improve the protection of data saved in a hearing system, e.g. sensitive health data of the user saved in his hearing device, so that even serious hackers may not be able to access this data. It is a further feature of the present technology to enable a secure sharing of the data saved in a hearing system with authorized third parties, such as physicians, who should have access to this data.

These features are achieved by the subject-matter of the independent claims. Further exemplary embodiments are evident from the dependent claims and the following description.

A first aspect of the present technology relates to a method for a protected sharing of data saved in a hearing system, which includes at least a hearing device used by a hearing device user, with a third party device. In general, a third party may be any person or organization other than the hearing device user. For example, the third party may be one or more of the following group of persons or organizations: a health or medical professional; a hearing care professional; a general practitioner; a medical specialist; a call center; a hearing care manufacturer, an emergency service or the public, who might need some data saved in the hearing device, such as health data of the hearing device user. A third party device may, for example, be a personal computer or a smartphone of the third party.

The method may be a computer-implemented method, which may be performed automatically by the hearing system. The hearing system as described herein below may be adapted for performing the method. The hearing system may, for instance, comprise one or two hearing devices used by the same user. One or both of the hearing devices may be worn on and/or in an ear of the user. A hearing device may be a hearing aid, which may be adapted for compensating a hearing loss of the user. Also a cochlear implant may be a hearing device. The hearing system may optionally further comprise at least one connected user device, such as a smartphone, smartwatch, tablet or other devices in particular bluetooth enabled, carried by the user.

According to an embodiment of the present technology, the method comprises: storing a definition of a first trusted party and of a second trusted party in the hearing system. Beside storing such a definition in a hearing device, this may also include storing a definition in a connected user device, such as a smartphone, and/or in a central server connected with the hearing device in a predefined way. A connected user device and/or the central server may be regarded as parts the hearing system.

The first trusted party and the second trusted party may, for example, be two different persons or organizations from the following group: the hearing device user himself; a health professional; a hearing care professional; a significant other, such as a spouse or another family member or other person close to the hearing device user; a hearing device manufacturer.

According to an embodiment of the present technology, the method further comprises: providing (e.g. transmitting or directing) a sharing request for the data to be shared to a device of the first trusted party (e.g. to a smartphone of the hearing device user) and to a device of the second trusted party (e.g. to a smartphone or personal computer of the significant other). The sharing request may, for instance, be generated by the user of the hearing device, using his smartphone or the hearing device. Alternatively, e.g. in an emergency case, the sharing request may also be generated by the third party, such as an emergency service.

According to an embodiment of the present technology, the method further comprises: generating a first sharing approval by the device of the first trusted party and a second sharing approval by the device of the second trusted party, and sharing the data with the third party device only upon generation of the first and the second sharing approval, i.e. only if both sharing approvals have been successfully generated. The above-mentioned step of storing a definition of the first trusted party and of the second trusted party in the hearing system may, as the case may be, also include storing a definition of a respective type of generation of a sharing approval or a computer-implemented algorithm of generating or validating the respective sharing approval.

In other words, according to the present method, at least two different predefined parties—the first and the second trusted party (and, as the case may be, a number of further trusted parties) whose definition is saved in the hearing system and/or a server connected to the hearing system—must authorize the sharing of data saved in the hearing system with a third party. If at least one of the sharing approvals of the trusted parties defined in the system is missing or fails, the sharing of data with the third party device will be prohibited and will not be performed. In this manner, the sharing of data saved in the hearing system can be reliably protected.

According to an embodiment of the present technology, different trusted parties may be authorized for different kinds of sharing approvals. A sharing approval may, for example, be restricted to a predetermined type of data to be shared, e.g. only authorized for approving the sharing of pulse data, but not of diagnosis data. Furthermore, respective sharing approvals may be restricted with respect to the receiver, i.e. the intended third party, e.g. only authorized for approving the sharing of data with medical practitioners, but not with insurance companies.

The sharing approvals may, as the case may be, provided with a time limit of their validity, so that data cannot be shared after the time limit, e.g. one month after data generation and/or after initiating the sharing request, has expired. It also may be that there is a spatial limit, i.e. that data sharing is only allowed at home, for example.

According to an embodiment of the present technology, the data to be shared is collected by a sensor which is a part of the hearing device and/or it may be derived from such sensor data (e.g. diagnostic data). It may be that one or more different sensors are integrated in the hearing device and/or transmit data measured at some other body part of the hearing device user by wireless communication to the hearing device.

In this sense, hearing devices provided with sensors may, for example, collect and store sensitive health data of one or more of the following types: pulse; body temperature; blood pressure; blood oxygen; blood sugar; electroencephalography (EEG) data; audiograms; falls and balance disorders; Dementia, Alzheimer's, Parkinson related data; Corona related data, e.g. concerning contact tracing, test, and vaccination.

The sensors may comprise one or more of the following: an air conduction microphone; a bone conduction microphone; an optical sensor; an electrical sensor; a heart-rate sensor; a blood pressure sensor; an electrocardiography (ECG), electroencephalography (EEG) or electrooculography (EOG) sensor.

However, the presence of a data collecting sensor is not indispensable for the present technology. For example, at least a part of the data to be shared may also be useful data manually saved in the hearing device, e.g. by a hearing care professional or by the hearing device user.

According to an embodiment of the present technology, the method further comprises: storing a definition of at least one further trusted party in the hearing system; providing a sharing request to a device of each further trusted party; and generating a further sharing approval by the device of each further trusted party. In this case, the data will be shared with the third party device only upon generation of the first sharing approval, the second sharing approval, and the at least one further sharing approval, i.e. only if further sharing approvals have been generated by the respective devices of all of the further trusted parties, too. By further increasing the number of trusted parties, the security provided by the present method may be further improved. All the details described above and in the following with respect to the first or the second trusted parties may pertain to each of the further trusted parties as well.

According to an embodiment of the present technology, storing a definition of the respective trusted party comprises storing its public key in the hearing system; and providing the sharing request to the device of the respective trusted party comprises encrypting the data to be shared with the public key of the respective trusted party and transmitting the encrypted data to its device. In this embodiment, the generation of the respective sharing approval by the device of the respective trusted party comprises decrypting the data to be shared with a private key of the respective trusted party and, for example, transmitting thus decrypted data to a predetermined receiving device of the hearing system for a next step of the present method.

With this embodiment, for example, a cascaded encryption may be implemented in that the data to be shared is encrypted with the public keys of several trusted parties and, as the case may be, additionally also with a public key of the third party. Thus encrypted data may be, first, transmitted to the device of the first trusted party, who can prove all the necessary details and approve the sharing by decrypting the data in its device with its private key and by transmitting thus decrypted data to the device of the second trusted party. The second trusted party can proceed in the same manner and transmit the data decrypted with its private key in its device to the device of a further trusted party or, if no further trusted parties are predefined in the hearing system, to the device of the third party, thus sharing the data with the third party. This may be seen as a four-eyes principle implemented by the method.

In this or any other embodiment which makes use of asymmetric encryption including a pair of keys, i.e. a public and a private key, alternatively, symmetric encryption may be implemented in the present method and system. The solution based on symmetric encryption may comprise the step of exchanging the symmetric key based on asymmetric encryption and/or based on a public key infrastructure. Further, in any embodiment described herein above or in the following which makes use of a public key, alternatively or in addition, a certificate including predefined additional information about the respective party may be used, for a still higher security of the method. The certificate may comprise a time limit.

Furthermore, any embodiment described herein above or in the following, which makes use of asymmetric encryption or public keys may also make use of a public key infrastructure including a registration and certification authority, such as e.g. a hearing device manufacture or an authority associated with the hearing device manufacture. Participating parties, such as the two or more trusted parties, on the one hand, and/or one or more third parties interested in the data to be shared, on the other hand, may deposit their public keys at the registration authority. From these, for example, certificates signed with a private key of the certification authority may be generated and offered for download over the Internet.

According to an embodiment of the present technology, storing a definition of the respective trusted party comprises storing its public key in the hearing system; and generating the respective sharing approval by the device of this trusted party comprises generating and signing a sharing permission with a private key of this trusted party and, for example, transmitting the signed sharing permission to a predetermined receiving device of the hearing system for a next step of the present method.

According to an embodiment of the present technology, storing a definition of the respective trusted party comprises storing its voiceprint in the hearing system; and generating the respective sharing approval by the device of this trusted party comprises transmitting a predetermined sharing approval phrase and/or keyword read and/or uttered by this trusted party as an audio signal from its device to a predetermined receiving device of the hearing system and verifying the received audio signal by comparing it with the saved voiceprint of the respective trusted party.

According to an embodiment of the present technology, storing a definition of the respective trusted party comprises storing its symmetric key in the hearing system; and providing the sharing request to the device of the respective trusted party comprises encrypting a sharing request with the symmetric key of this trusted party and transmitting thus encrypted sharing request to its device. In this embodiment, the generation of the respective sharing approval by the device of this trusted party comprises generating and encrypting a sharing approval with the symmetric key of this trusted party and, for example, transmitting thus encrypted sharing approval to a predetermined receiving device of the hearing system for a next step of the present method. By using symmetric keys instead of asymmetric keys, memory space and/or calculation capacity of the hearing system may be spared.

According to an embodiment of the present technology, the method further comprises: storing connection data (e.g. a URL) for connecting to a predetermined central server as well as a public key of this central server in the hearing system. In this embodiment, storing a definition of the respective trusted party comprises storing an identification number (ID) and/or a public key of the respective trusted party in the central server; and providing the sharing request to the device of the respective trusted party comprises transmitting the sharing request from the central server to the device of the respective trusted party. This may, for instance, include providing a sharing request to said central server, whereupon it transmits the sharing request to the device of the respective trusted party. Further, in this embodiment, generating the respective sharing approval may comprise transmitting a signed sharing permission signed with a private key of the respective trusted party from its device to the central server.

With this embodiment, all authorizations may be centrally managed by the central server. Here, instead of storing public keys of trusted parties in the hearing device, for example, only an ID of the respective trusted party (e.g. the first or second trusted party) and the public key and the URL of the central server may be saved in the hearing device or elsewhere in the hearing system. Using this ID, the public key of the respective trusted party can be retrieved from the central server. The respective public key may, for example, be signed with a private key of the central server. The ID may comprise, by way of example only, one or more of the following: a random number; a phone number; a name; a customer number; a social insurance number of the respective party.

According to an embodiment of the present technology, the method further comprises: encrypting the data to be shared with a public key or with a symmetric key of the third party, prior to sharing the data. Thereby, the security of the method and system may be further increased.

According to an embodiment of the present technology, the step of generating the first and/or second sharing approval further comprises obtaining and checking a predefined context information comprising one or more of the following: data regarding the third party (in particular the name and profession of the third party); the kind of data to be shared; the time when the data was collected; the time when the sharing was requested (e.g. by the hearing device user or by the third party, such as an emergency service), i. e. the time the sharing request was generated or provided to the trusted parties; a time when the sharing can be done the earliest; a time when the sharing can be done the latest; a location of the hearing device from which the sharing is allowed, e.g. at home; a number and/or kind of trusted parties; a required kind of biometric data; and/or a required kind of out-of band response. In this embodiment, as the case may be, data regarding the third party and/or a further party may be digitally signed, too.

According to an embodiment of the present technology, the hearing system is configured such that the step of storing a definition of the respective trusted party is restricted to be performed only by a predetermined defining authority, whose connection data (e.g. a URL) and/or public key are saved in the hearing system. The defining authority may, for instance, be a superordinate defining authority, such as the manufacturer of the hearing device. It may also be that the superordinate defining authority may set an intermediary defining authority, e.g. a hearing professional. This may be done during fitting of the hearing device. In this case, the superordinate defining authority passes its authorization to save a definition of the trusted parties to the intermediary defining authority, e.g. by issuing a signed authorization, which may be signed with a private key of the superordinate defining authority and comprise the public kea of the intermediary defining authority. In the hearing device or system, a validation of this authorization may be implemented, e.g. by proving the signature of the authorization using the public key of the superordinate defining authority saved in the hearing system. It also may be that the authorization as the defining authority is provided with a time limit of validity. It may also be that two or more independent defining authorities are provided, e.g. such that at least two different trusted parties can only be defined by at least two different defining authorities. For example, a hearing care professional and his costumer do this together.

According to an embodiment of the present technology, the hearing system is configured such that the steps of storing a definition of the respective trusted party and/or of generating the respective sharing approval are at least partly restricted to a predetermined out-of-band-communication channel (such as only via a microphone of the hearing device by using a voice recognition software and/or acoustically encoded data such as DTMF, dual-tone multi-frequency, or only via predetermined wired or wireless communication channels, e.g. a wired interface, Bluetooth, WLAN, a conventional telephone service, landline, cellular, GSM (3G, 4G, 5G), SMS Service, e-Mail and/or the Internet), which is different from a data communication channel used for sharing the data saved in the hearing system with the third party device.

Further aspects of the present technology relate to a computer program for a protected sharing of data saved in a hearing system, which system includes at least a hearing device used by a hearing device user, with a third party device, which program, when being executed by a processor, is adapted to carry out the steps of the method as described above and in the following as well as to a computer-readable medium, in which such a computer program is stored. The computer program may have several parts running in different device, i.e. may be executed in a distributed environment.

For example, the computer program may be executed in a processor of a hearing device, which hearing device, for example, may be carried by a person, i.e. the user, behind the ear. The computer-readable medium may be a memory of this hearing device. The computer program also may be executed by a processor of a connected user device, such as a smartphone or any other type of mobile device, which may be a part of the hearing system, and the computer-readable medium may be a memory of the mobile device. It also may be that some steps of the method are performed by the hearing device and other steps of the method are performed by the mobile device. In general, some of the steps may be performed in a first device and other steps may be performed in a second device.

In general, a computer-readable medium may be a floppy disk, a hard disk, an USB (Universal Serial Bus) storage device, a RAM (Random Access Memory), a ROM (Read Only Memory), an EPROM (Erasable Programmable Read Only Memory) or a FLASH memory. A computer-readable medium may also be a data communication network, e.g. the Internet, which allows downloading a program code. The computer-readable medium may be a non-transitory or transitory medium.

A further aspect of the present technology relates to a hearing system comprising at least one hearing device, a device of a first trusted party and a device of a second trusted party, wherein the hearing system is adapted for performing the method described herein above and below. Apart from the hearing device mentioned above, the hearing system may further include, by way of example, a second hearing device used by the same user and/or a connected user device, such as a smartphone or other mobile device or personal computer, used by the same user and/or a central server connected with the hearing device in a predefined way over a data communication network, e.g. the Internet.

According to an embodiment of the present technology, the hearing device comprises: a microphone; a processor for processing a signal from the microphone; an output device for outputting the processed signal to an ear of the hearing device user; and a transceiver for exchanging data with the device of the first and/or second trusted party and/or with a connected user device and/or with a central server and/or with another hearing device.

The definition of the two or more trusted parties of the present method may be, for example, saved in the hearing device by programming. As described in more detail above, the programming may be performed e.g. by storing a public key of the respective trusted party in the hearing device. As also described above, the public key may be a part of a certificate of the respective party. Furthermore, a time limit may be provided for the validity of the respective public key or certificate.

The definition of the two or more trusted parties may, for instance, be performed during a first fitting session, e.g. in an office of a hearing professional, where the at least one hearing device is being fitted to its user. In the course of this, the hearing device may, for instance, be connected with a fitting computer (serving as an intermediary defining authority), e.g. by wireless communication such as Bluetooth. The fitting computer, in its turn, may be connected over a data communication network such as the Internet with a superordinate defining authority, such as the manufacturer of the hearing device.

It may also be implemented that the hearing device user may change the definition of the trusted parties without visiting the office of the hearing professional again. To this end, for example, the public key of the hearing professional or another defining authority may be saved in the hearing device or system. This may be in the context of a so called “remote fitting” where an audiologist can remotely program a hearing aid (tele audiology). It may also be in the context of a self-fitting where the hearing aid user adjusts the hearing aid by him- or herself.

Using the public key of the defining authority, the user may effect a redefinition of trusted parties in a verified manner, in that the corresponding programming commands transmitted from the defining authority to the hearing system are signed with the private key of the defining authority.

Depending on the security requirements, the present technology, e.g. the steps of the method described herein, may be primarily implemented in the hearing device or, alternatively, in a mobile application running on another device of the hearing system, e.g. on a smartphone (via a corresponding Smartphone App) or another connected user device. The more of the steps and aspects of the present technology are implemented in the hearing device, the higher level of protection for the data sharing against unauthorized hacking might be achievable. To this end, in particular, a so-called Trusted Platform Module may be a part of the hearing device, e.g. such as disclosed in EP 3 236 674 A1 as described at the outset. By a Trusted Platform Module, the hearing device may be, for instance, provided with public key security functions. That is, a Trusted Platform Module may include a secure cryptoprocessor, a random generator, as well as a protected memory for private keys, which is only readable by the cryptoprocessor, but not by the main processor. The public key of the device, the public key of a hearing device manufacturer and an operating system may be saved in a storage region of a non-volatile memory of the hearing device which is protected against modification, i.e. in a sealed storage region.

It has to be understood that features of the method as described above and in the following may be features of the computer program, the computer-readable medium and the hearing system as described above and in the following, and vice versa.

These and other aspects of the present technology will be apparent from and elucidated with reference to the embodiments described hereinafter.

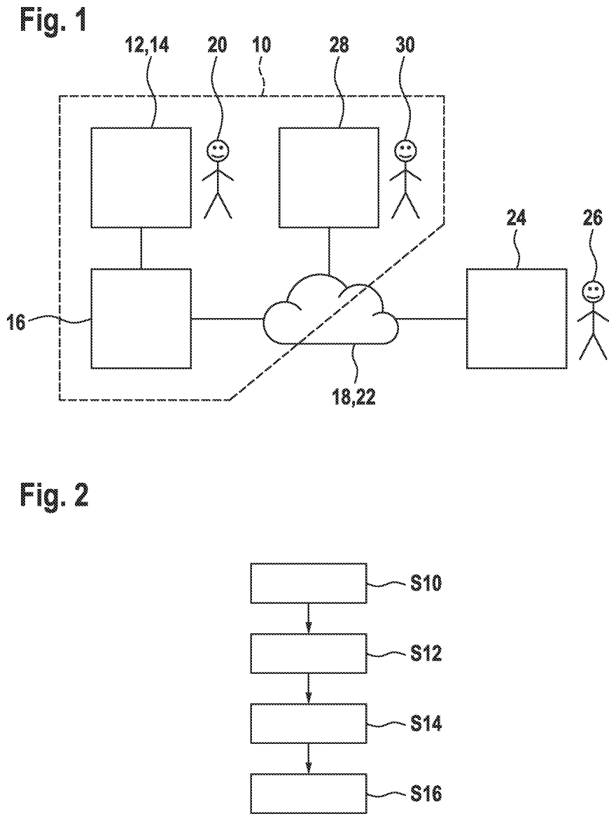

FIG. 1 shows a hearing system 10 which comprises two hearing devices 12, 14 and a connected user device 16.

Each of the two hearing devices 12, 14 may be worn by a user 20 behind the ear and/or in the ear. For example, the hearing device 12 may be a left hearing device for the left ear and the hearing device 14 may be a right hearing device for the right ear. The two hearing devices 12, 14 may constitute a binaural hearing system. The hearing devices 12, 14 may be hearing aids adapted for compensating a hearing loss of the user 20. At least one of the hearing devices 12, 14 may comprise one or more sensors, e.g. accelerometers, pulse oximetry or electroencephalography sensors, collecting health data of the user 20. The two hearing devices 12, 14 may be adapted for exchanging data wirelessly, for example via Bluetooth (e.g. BLE, Low Energy). For example, it may be that an audio signal received and/or processed by the first hearing device 12 and/or sensor data collected by its sensors may be transmitted to the second hearing device 14, and vice versa.

The connected user device 16, which may be a tablet computer or smartphone, also may be adapted for exchanging data wirelessly with one or both of the hearing devices 12, 14, for example via Bluetooth. Furthermore, the hearing devices 12, 14 and/or the user device 16 may be adapted for exchanging data with one or more data communication networks 18, such as the Internet and/or a telephone network. Via the data communication networks 18, the hearing devices 12, 14 and/or the user device 16 may also be able to connect to a predetermined remote central server 22, which may also be regarded as a part of the hearing system 10, as schematically indicated by a dashed line in FIG. 1.

The hearing system 10 is adapted for performing a method according to the present technology for a protected sharing of data saved in the hearing system 10, e.g. health data of the user 20 saved in the hearing devices 12 and 14, with a device 24, such as a personal computer, of a third party 26, e.g. a health professional or an emergency service, who might need this data to help the user 20 in a health treatment or an emergency case. To this end, the hearing system 10 further comprises a device 28, e.g. a mobile device such as a smartphone, of a significant other 30, who may, for example, be a spouse or another family member of the user 20.

FIG. 2 shows a flow diagram for a method for a protected sharing of data saved in the hearing system 10 with the third party device 24.

A first step S10 of the method may be performed, for instance, during a fitting session in an office of a hearing professional, where the hearing devices 12 and 14 are fitted to their user 20. In step S10, storing a definition of a first trusted party and of a second trusted party in the hearing system 10 is performed. Beside storing such a definition in a hearing device 12 or 14, this may also include storing the definition in the connected user device 16 and/or in the central server 22. In order to save the definition of the trusted parties, the hearing device 12 and/or 14 may, for instance, be connected with a fitting computer (serving as an intermediary defining authority), e.g. by wireless communication such as Bluetooth.

In the present example, the first trusted party is the hearing device user 20, whereas the second trusted party is a significant other 30, such as his/her spouse.

In step S12, a sharing request for the data to be shared is provided to the device 16 of the first trusted party (in this example to the smartphone of the hearing device user 20) and to the device 28 of the second trusted party (in this example to a smartphone or personal computer of the significant other 30). The sharing request may, for instance, be generated by the user 20 himself, using his smartphone or one of the hearing devices 12 or 14. Alternatively, e.g. in an emergency case, the sharing request may also be generated by the third party 26, such as an emergency service.

In step S14, a first sharing approval is generated by the device 16 of the first trusted party and a second sharing approval is generated by the device 28 of the second trusted party.

In final step S16, sharing of the data with the third party device 24 is only performed, if both sharing approvals have been successfully generated.

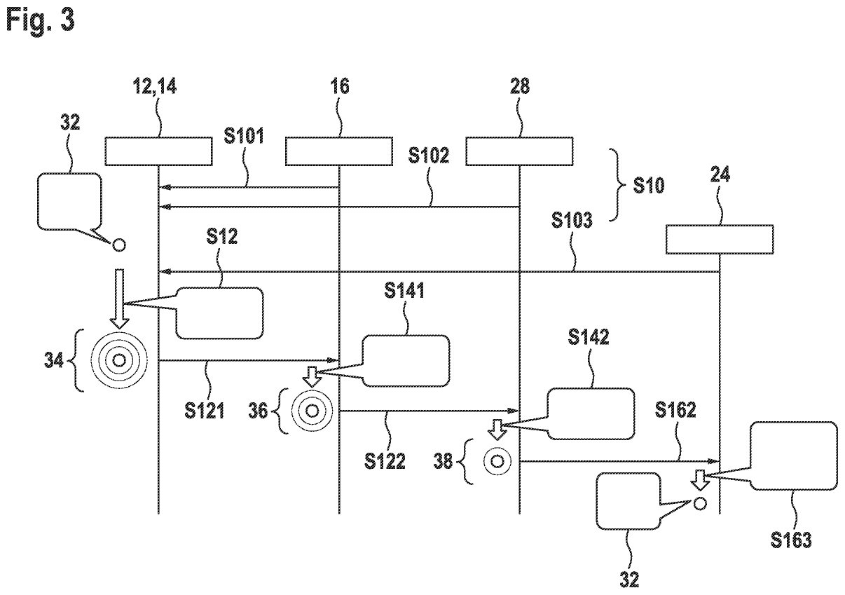

A number of possible implementations of the steps S10 to S16 will be visualized in the following with reference to FIGS. 3 to 6.

FIG. 3 schematically shows a block diagram visualizing the method of FIG. 2 according to a first embodiment of the present technology, involving cascaded encryption. In this embodiment, storing a definition of the respective trusted party in step S10 comprises storing S101, S102 its public key in the hearing device 12 or 14. Furthermore, providing the sharing request to the device 16, 28 of the respective trusted party in step S12 comprises encrypting plain sensor data 32 collected and saved in the hearing device 12 or 14 with the public key of the respective trusted party and transmitting S121, S122 thus encrypted data 34 to its respective device 16, 28. Further, the generation of the respective sharing approval by the device 16, 28 of the respective trusted party in step S14 comprises decrypting S141, S142 the data with a private key of the respective trusted party and transmitting thus decrypted data 36, 38 to a predetermined receiving device of the hearing system 10 for a next step of the present method.

In other words, as illustrated in FIG. 3, a cascaded encryption may be implemented by this embodiment. That is, plain sensor data 32, which is collected and saved in the hearing device 12 or 14 and is to be shared with the third party 26, is encrypted in step S12 with the public key of the first trusted party, with the public key of the second trusted party and, optionally, also with a public key of the third party 26 previously obtained in an additional step S103. Thus encrypted data 34 is transmitted in substep S121 to the device 16 of the first trusted party, who can prove all the necessary details and approve the sharing by decrypting S141 the data 34 in its device 16 with its private key and by transmitting S122 thus decrypted data 36 to the device 28 of the second trusted party. The second trusted party can proceed in the same manner and transmit S162 the data 38 decrypted S142 with its private key in its device 28 to the device 24 of the third party 26, thereby sharing this data with the third party 26. In this example, the shared data 38 is, finally, decrypted S163 by the third party 26 with its private key in its device 24, which renders plain sensor data 32, whereby the sharing step S16 is completed.

The method illustrated in FIG. 3 may, in particular, comprise the following steps:

Storing a first public key of a first trusted party in the hearing device 12, 14

Storing a second public key of a second trusted party in the hearing device 12, 14

Acquiring sensor data with the sensors

Analyzing the sensor data in the hearing device 12, 14

Reporting a health condition to the hearing device user 20

Selecting a third party 26 (e.g. medical professional) by the hearing device user 20

Generating by the connected user device 16 a request to share the data with the third party (e.g. using a suitable app running on the device 16); the hearing device 12, 14 receiving this sharing request

Obtaining a public key of the third party 26 from the third party device 24

Encrypting the data in the hearing device 12, 14 with the public key of the third party 26

Encrypting the data in the hearing device 12, 14 with the first public key

Encrypting the data in the hearing of the third party 26 with the second public key

Transmitting the data from the hearing device to the device 16 of the first trusted party

Decrypting the data by the first trusted party in its device 16

Transmitting the data from device 16 of the first trusted party to the device 28 of the second trusted party

Decrypting the data by the second trusted party in its device 28

Transmitting the data from the device 28 of the second trusted party to the device 24 of the third party 26

Decrypting the shared data by the third party 26 in its device 24.

Encrypting the data in the hearing device 12, 14 with the first public key and encrypting the data in the hearing of the third party 26 with the second public key sets the order of decryption. However it is also possible to generate two sets of data, which are encrypted with the first public key and with the second pubic key in different orders.

Furthermore, the data may comprise meta information, which medical data are encoded into the data, who should receive the data. The meta information may be added to the data after the first encrypting step, such that the corresponding trusted party is able to read the meta information after the first decryption step without the need for performing the second decryption step.

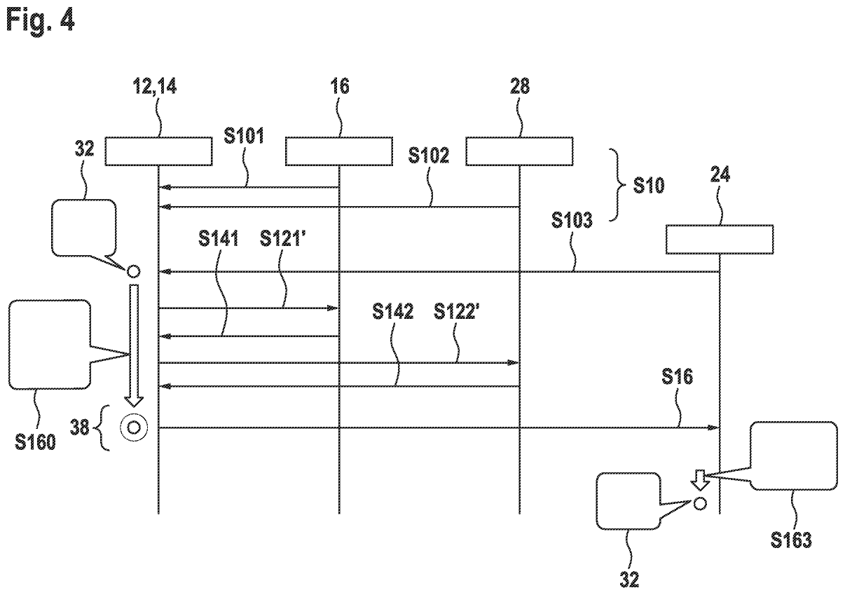

FIG. 4 schematically shows a block diagram visualizing the method of FIG. 2 according to a second embodiment of the present technology involving signed sharing permissions. In this embodiment, storing a definition of the respective trusted party in step S10 comprises storing S101, S102 its public key in the hearing device 12 or 14; and providing the sharing request to the device 16, 28 of the respective trusted party in step S12 simply comprises transmitting S121′, S122′ the sharing request to its respective device 16, 28 from the hearing device 12, 14. Further, the generation of the respective sharing approval by the device 16, 28 of the respective trusted party in step S14 comprises generating a sharing permission signed with a private key of this trusted party and transmitting S141, S142 the signed sharing permission to a predetermined receiving device of the hearing system 10, such as to the hearing device 12 or 14, for a next step of the present method, i.e. for the step S16 of sharing the data. Similarly to FIG. 3, prior to be shared, the plain sensor data 32 is encrypted in step S160 with a public key of the third party 26 obtained from its device 24 in step S103. The shared encrypted data 38 may then be decrypted by the third party 26 in its device 24 in step S163, so as to obtain plain sensor data 32 and/or data derived from it, e.g. diagnostic data, and, thus, complete the sharing.

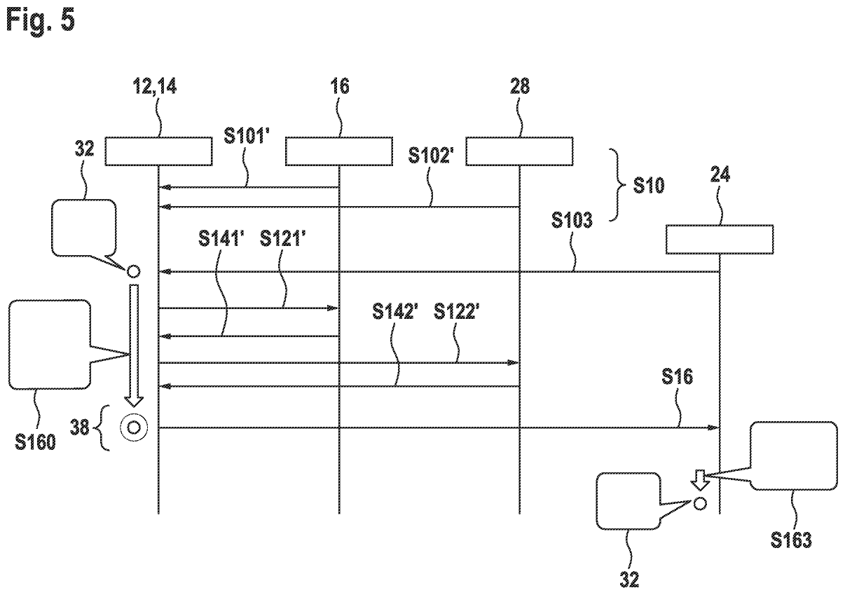

FIG. 5 schematically shows a block diagram visualizing the method of FIG. 2 according to a third embodiment of the present technology involving voiceprints of trusted parties. This embodiment only differs from the one shown in FIG. 4 in that storing a definition of the respective trusted party in step S10 comprises storing its voiceprint S101′, S102′ in the hearing device 12 or 14; and in that the generation of the respective sharing approval by the device 16, 28 of the respective trusted party in step S14 comprises transmitting S141′, S142′ a predetermined sharing approval phrase read by this trusted party as an audio signal from its device 16, 28 to the hearing device 12 or 14 and verifying the received audio signal by comparing it with the saved voiceprint of the respective trusted party. It also may be that instead of the complete audio signal solely features of the audio signal are transmitted. The analysis of the audio signal may be performed in a server. The audio signal may be transmitted “out of band”, for example via the hearing device microphone and/or via the classical telephone network. Alternatively, or in addition to a voice print there may be other biometric methods, such as iris scan and/or fingerprint.

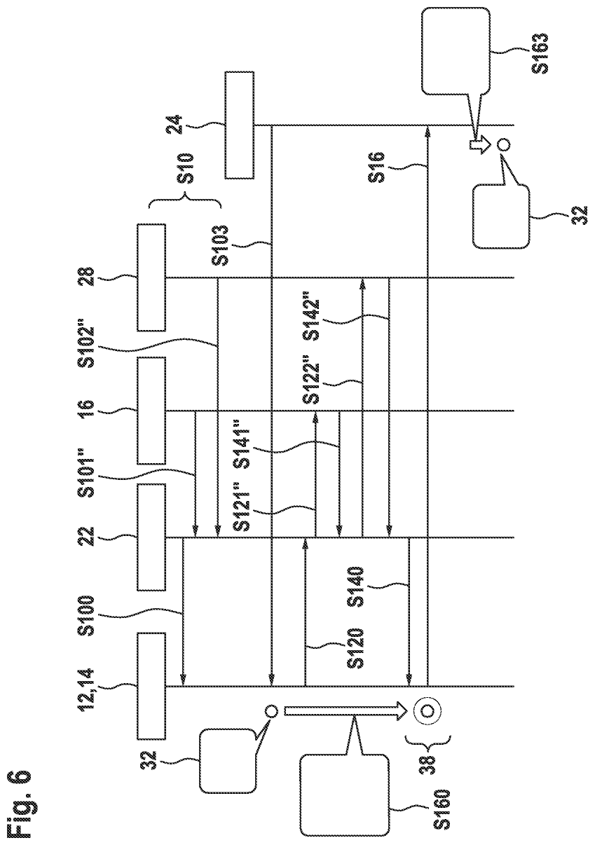

FIG. 6 schematically shows a block diagram visualizing the method of FIG. 2 according to a fourth embodiment of the present technology involving a central server managing the sharing approvals. According to this embodiment of the present technology, the method further comprises a step S100 of storing connection data (e.g. an ID and/or a URL) for connecting to a predetermined central server 22 as well as a public key of this central server 22 in the hearing system 10, e.g. in the hearing device 12 or 14.

Further, in FIG. 6, storing a definition of the respective trusted party in step S10 comprises storing S101″, S102″ an ID and/or a public key of the respective trusted party in the central server 22. Further, providing the sharing request to the device 16, 28 of the respective trusted party in step S12 comprises providing S120 a sharing request to the central server 22, whereupon it transmits S121″, S122″ the sharing request to the device 16, 28 of the respective trusted party. Further, in this embodiment, the step S14 of generating the respective sharing approval comprises transmitting S141″, S142″ a signed sharing permission signed with a private key of the respective trusted party from its device 16, 28 to the central server 22, whereupon the central server 22 transmits S140 a signed sharing permission signed with a private key of the central server 22 to the hearing device 12 or 14. Similarly to FIGS. 4 and 5, prior to be shared, the plain sensor data 32 is encrypted in step S160 with a public key of the third party 26 obtained from its device 24 in step S103. The shared data 38 may then also be decrypted by the third party 26 in its device 24 in step S163, so as to obtain plain sensor data 32 and, thus, complete the sharing.

With this embodiment, all authorizations may be centrally managed by the central server. Here, instead of storing public keys of trusted parties in the hearing device, for example, only an ID of the respective trusted party (e.g. the first or second trusted party) and the public key and the URL of the central server may be saved in the hearing device 12, 14 or in the central server 22 of the hearing system 10. Using this ID, the public key of the respective trusted party can be retrieved from the central server 22. The respective public key may, for example, be signed with a private key of the central server. The ID may comprise, by way of example only, one or more of the following: a random number; a phone number; a name; a customer number; a social insurance number.

The examples of FIGS. 3 to 6 apply asymmetric encryption, private and public keys likewise. They may also be embodied fully or partially based on symmetric encryption.

While the present technology has been illustrated and described in detail in the drawings and foregoing description, such illustration and description are to be considered illustrative or exemplary and not restrictive; the present technology is not limited to the disclosed embodiments. Other variations to the disclosed embodiments can be understood and effected by those skilled in the art and practicing the claimed present technology, from a study of the drawings, the disclosure, and the appended claims. In the claims, the word “comprising” does not exclude other elements or steps, and the indefinite article “a” or “an” does not exclude a plurality. A single processor or controller or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

### LIST OF REFERENCE SYMBOLS

10 hearing system
12 first hearing device
14 second hearing device
16 connected user device, device of a first trusted party
18 data communication network(s)
20 hearing device user, first trusted party
22 central server
24 device of a third party
26 third party, with whom data is to be shared
28 device of a second trusted party
30 significant other, second trusted party
32 plain sensor data
34 sensor data encrypted with the public keys of the first and the second trusted party, as well as with the public key of the third party
36 sensor data only encrypted with the public keys of the second trusted party and of the third party
38 sensor data only encrypted with the public key of the third party
S10 storing a definition of a first trusted party and of a second trusted party in the hearing system
S12 providing a sharing request to the device of the first trusted party and to the device of the second trusted party
S14 generating a first sharing approval by the device of the first trusted party and a second sharing approval by the device of the second trusted party
S16 sharing of the data with the third party device
S101 storing a public key of the first trusted party in the hearing device
S102 storing a public key of the second trusted party in the hearing device
S101′ storing a voiceprint of the first trusted party in the hearing device
S102′ storing a voiceprint of the second trusted party in the hearing device
S101″ storing an ID and/or a public key of the first trusted party in the central server
S102″ storing an ID and/or a public key of the second trusted party in the central server
S100 storing connection data and/or a public key of the central server in the hearing system
S103 storing a public key of the third party in the hearing device
S120 providing a sharing request to the central server
S121′ transmitting a sharing request to the device of the first trusted party from the hearing device
S122′ transmitting a sharing request to the device of the second trusted party from the hearing device
S121″ transmitting a sharing request to the device of the first trusted party from the central server
S122″ transmitting a sharing request to the device of the second trusted party from the central server
S121 encrypting the data to be shared with the public key of the first trusted party and transmitting it, as a sharing request, to its device
S122 encrypting the data to be shared with the public key of the second trusted party and transmitting it, as a sharing request, to its device
S141 decrypting the data with the private key of the first trusted party, or transmitting a sharing permission signed therewith to the hearing device
S142 decrypting the data with the private key of the second trusted party, or transmitting a sharing permission signed therewith to the hearing device
S141′ transmitting a predetermined sharing approval phrase read by the first trusted party as an audio signal from its device and verifying the received audio signal by comparing it with the saved voiceprint
S142′ transmitting a predetermined sharing approval phrase read by the second trusted party as an audio signal from its device and verifying the received audio signal by comparing it with the saved voiceprint
S141″ transmitting a sharing permission signed with the private key of the first trusted party to the central server
S142″ transmitting a sharing permission signed with the private key of the second trusted party to the central server
S140 transmitting a signed sharing permission signed with a private key of the central server to the hearing device
S160 encrypting data to be shared with a public key of the third party
S163 decrypting the shared data with a private key of the third party in its device

What is claimed is:

1. A method for a protected sharing of data stored in a hearing system, which comprises at least a hearing device used by a hearing device user, with a third party device, the method comprising:
storing a definition of a first trusted party and of a second trusted party in the hearing system;
providing a sharing request for the data to be shared to a device of the first trusted party and to a device of the second trusted party;
generating a first sharing approval by the device of the first trusted party and a second sharing approval by the device of the second trusted party, and sharing the data with the third party device upon generation of the first and the second sharing approval.

2. The method of claim 1,
wherein the data to be shared is collected by a sensor and/or is derived from data collected by a sensor, which is a part of the hearing device.

3. The method of claim 1, further comprising:
storing a definition of at least one further trusted party in the hearing system;
providing a sharing request to a device of each further trusted party; and
generating a further sharing approval by the device of each further trusted party, and sharing the data with the third party device upon generation of the first sharing approval, the second sharing approval, and the at least one further sharing approval.

4. The method of claim 3, wherein:
the storing the definition of the respective trusted party comprises storing a public key in the hearing system;
the providing the sharing request to the device of the respective trusted party comprises encrypting the data to be shared with the public key of the respective trusted party and transmitting the encrypted data to the device; and
the generating the respective sharing approval comprises decrypting the data to be shared with a private key of the respective trusted party in the device.

5. The method of claim 3, wherein:
the storing the definition of the respective trusted party comprises storing a public key of the respective trusted party in the hearing system; and
the generating the respective sharing approval comprises signing a sharing permission with a private key of the respective trusted party in the device.

6. The method of claim 3, wherein:
storing a definition of the respective trusted party comprises storing a voiceprint in the hearing system; and
generating the respective sharing approval comprises transmitting a predetermined sharing approval phrase and/or keyword, read and/or uttered by the respective trusted party as an audio signal from the device of the respective trusted party to a predetermined receiving device of the hearing system and verifying the received audio signal by comparing it with the voiceprint of this trusted party.

7. The method of claim 3, wherein:
the storing the definition of the respective trusted party comprises storing a symmetric key in the hearing system;
the providing the sharing request to the device of the respective trusted party comprises encrypting a sharing request with the symmetric key of this trusted party and transmitting thus encrypted sharing request to the device; and
the generating the respective sharing approval comprises encrypting a sharing approval with the symmetric key of the respective trusted party in the device.

8. The method of claim 3, wherein:
connection data for connecting to a predetermined central server as well as a public key of the predetermined central server are saved in the hearing system;
the storing the definition of the respective trusted party comprises storing an identification number and/or a public key of the respective trusted party in said central server;
providing the sharing request to the device of the respective trusted party comprises transmitting the sharing request from said central server to the device of the respective trusted party; and
generating the respective sharing approval comprises transmitting a signed sharing permission signed with a private key of the respective trusted party from the device to said central server.

9. The method of claim 1,
wherein, prior to be shared, the data is encrypted with a public key of the third party or with a symmetric key of the third party.

10. The method of claim 1, wherein the generating the first and/or second sharing approval comprises obtaining and checking a predefined context information comprising one or more of:
data regarding the third party;
a kind of data to be shared;
a time when the data was collected;
a time when the sharing request was generated;
a time when the sharing can be done the earliest;
a time when the sharing can be done the latest;
a location of the hearing device from which the sharing is allowed;
a number and/or kind of trusted parties;
a required kind of biometric data; or
a required kind of out-of band response.

11. The method of claim 1, wherein the hearing system is configured such that the storing the definition of the respective trusted party is restricted to be performed only by a predetermined defining authority, the predetermined defining authority having connection data and/or a public key that are saved in the hearing system.

12. The method of claim 1, wherein the hearing system is configured such that the storing the definition of the respective trusted party and/or the generating the respective sharing approval are at least partly restricted to a predetermined out-of-band-communication channel, the predetermined out-of-band-communication channel different from a data communication channel used for sharing the data with the third party device.

13. A computer program for a protected sharing of data saved in a hearing system, which system comprises at least a hearing device used by a hearing device user, with a third party device, which program, when being executed by a processor, is adapted to carry out the steps of the method claim 1.

14. The computer program of claim 13, wherein the computer program is stored on a non-transitory computer-readable medium.

15. A hearing system comprising a hearing device, a device of a first trusted party and a device of a second trusted party, wherein the hearing device comprises:
a microphone;
a processor for processing a signal from the microphone;
an output device for outputting the processed signal to an ear of a hearing device user;
a transceiver for exchanging data with the device of the first and/or second trusted party and/or with a connected user device and/or with a central server and/or with another hearing device,
wherein the hearing system is adapted for performing the method claim 1.
